(19) United States
(12) Patent Application Publication
(10) Pub. No.: US 2023/0320794 A1
(43) Pub. Date: Oct. 12, 2023

(54) CONFIGURING A SURGICAL ROBOTIC SYSTEM

(71) Applicant: CMR Surgical Limited, Cambridge (GB)

(72) Inventors: Andrew Murray SCHOLAN, Hertfordshire (GB); Luke David Ronald HARES, Cambridge (GB)

(21) Appl. No.: 17/995,242

(22) PCT Filed: Mar. 29, 2021
(86) PCT No.: PCT/GB2021/050767

§ 371 Date: Sep. 30, 2022

(30) Foreign Application Priority Data
Mar. 31, 2020 (GB) 2004754.4

(51) Int. Cl. A61B 34/30; A61B 34/00; A61B 90/00

(52) U.S. Cl. CPC A61B 34/30; A61B 34/74; A61B 90/361; A61B 2034/302; A61B 2090/066

(57) ABSTRACT

A control system of a surgical robotic system, the surgical robotic system comprising a first robot arm and a second robot arm, each of the first and second robot arms comprising a series of joints by which the configuration of that robot arm can be altered, the series of joints extending from a base at a proximal end of the robot arm to an attachment for a surgical instrument at a distal end of the robot arm, the control system being configured to reconfigure the surgical robotic system by: controlling the first robot arm to operate in a surgical mode in which a first surgical instrument attached to that first robot arm is inside a patient's body; and whilst the first robot arm is operating in the surgical mode: (i) controlling the second robot arm so as to permit a second surgical instrument attached to the second robot arm to be inserted into a port in the patient's body; (ii) determining a fulcrum about which the second surgical instrument pivots when the configuration of the second robot arm is altered whilst the second surgical instrument is inside the port; and (iii) controlling the second robot arm to operate in a surgical mode in which the configuration of the second robot arm and second surgical instrument is controlled in response to inputs received at a remote surgeon console whilst maintaining an intersection between the second surgical instrument and the determined fulcrum.

### BACKGROUND

This invention relates to a method of reconfiguring a surgical robotic system.

Invasive medical procedures can be performed using surgical robotic systems. FIG. 1 shows a typical surgical robotic system. The surgical robotic system 100 is shown performing an invasive medical procedure on a patient 102 positioned on an operating table 103. The surgical robotic system 100 comprises three arms 101a, 101b and 101c. The three arms 101a, 101b, 101c attach to a common unit 110. Each arm 101a, 101b and 101c may carry a surgical tool 106, such as a tool for performing cutting or grasping or an imaging device such as an endoscope. Each arm 101a, 101b, 101c may manipulate the surgical tool that it carries in order to perform aspects of the invasive procedure. The surgical robotic system is supported by a base 109 resting on the floor of the operating room.

In the event that one or more of arms 101a, 101b or 101c develop a fault, or are no longer useable for any other reason, often a decision must be made as to whether: (i) the invasive procedure can be completed using only the remaining arms of the surgical robotic system; or (ii) the invasive procedure should be converted to a manual procedure (e.g. in which a surgeon, rather than the robotic surgical system, manipulates surgical tools in order to complete the procedure). Both of these scenarios are undesirable—both lead to an increased difficulty in completing the invasive procedure, and converting to a manual procedure often leads to a longer recovery time for the patient.

Thus, it would be desirable if there were an improved method of reconfiguring a surgical robotic system such that the abovementioned problems can be addressed.

### SUMMARY OF THE INVENTION

According to a first aspect of the invention there is provided a method of reconfiguring a surgical robotic system, the surgical robotic system comprising a first robot arm and a second robot arm, each of the first and second robot arms comprising a series of joints by which the configuration of that robot arm can be altered, the series of joints extending from a base at a proximal end of the robot arm to an attachment for a surgical instrument at a distal end of the robot arm, the method comprising: operating the first robot arm in a surgical mode in which a first surgical instrument attached to that first robot arm is inside a patient's body; and whilst operating the first robot arm in the surgical mode: (i) inserting a second surgical instrument attached to the second robot arm into a port in the patient's body; (ii) determining a fulcrum about which the second surgical instrument pivots when the configuration of the second robot arm is altered whilst the second surgical instrument is inside the port; and (iii) operating the second robot arm in a surgical mode in which the configuration of the second robot arm and second surgical instrument is controlled by the remote surgeon console whilst maintaining an intersection between the second surgical instrument and the determined fulcrum.

The fulcrum may be determined by: enabling the configuration of the second robot arm to be altered in response to external forces; applying external forces to the second robot arm such that its configuration is altered whilst the second surgical instrument is inside the port; and determining the fulcrum, the fulcrum being the point about which the surgical instrument of the second robot arm pivots whilst inside the port.

The second robot arm may further comprise one or more force sensors configured to sense forces at one or more joints of the series of joints of the second robot arm, and one or more motors configured to drive one or more joints of the series of joints of the second robot arm, and the method may further comprise: sensing, using the one or more force sensors, external forces at one or more joints of the series of joints of the second robot arm; driving, using the one or more motors, one or more joints of the series of joints of the second robot arm in dependence on the sensed external forces so as to alter the configuration of the second robot arm.

The second robot arm may further comprise one or more position sensors configured to sense the position of one or more joints of the series of joints of the second robot arm, and the method may further comprise: recording, using the at least one position sensor, the position of one or more joints of the series of joints of the second robot arm at a plurality of instances whilst the configuration of the second robot arm is being altered; determining, for each instance, a position of the distal end of the second robot arm in dependence on the respective recorded one or more joint positions; determining, for each instance, a vector of the second surgical instrument from the determined position of the distal end of the second robot arm in dependence on the respective recorded one or more joint positions; and determining the point of intersection of the determined vectors of the second surgical instrument so as to determine the fulcrum.

The fulcrum may be the natural rotation centre of the port.

The method may further comprise: determining the fulcrum when the second robot arm is operating in a calibration mode; and causing the second robot arm to transition from operating in the calibration mode to operating in the surgical mode.

The second robot arm may further comprise a more distal interface and a less distal interface, and the method may further comprise: causing the second robot arm to transition from operating in the calibration mode to operating in the surgical mode using the more distal interface.

The method may further comprise: inserting the second surgical instrument into the port by operating the second robot arm in a compliant mode in which the configuration of the second robot arm can be altered in response to external forces; and causing the second robot arm to transition from operating in the compliant mode to operating in the calibration mode.

The method may further comprise: causing the second robot arm to transition from operating in the compliant mode to operating in the calibration mode using the more distal interface.

The method may further comprise: after determining the fulcrum, operating the second robot arm in an instrument adjust mode in which the configuration of the second robot arm can be altered in response to external forces but is constrained such that an intersection is maintained between the second surgical instrument and the determined fulcrum; and applying external forces to the second robot arm such that its configuration is altered whilst maintaining an intersection between the second surgical instrument and the determined fulcrum.

The method may further comprise: causing the second robot arm to transition from operating in the calibration mode to operating in the instrument adjust mode; and causing the second robot arm to transition from operating in the instrument adjust mode to operating in the surgical mode.

The method may further comprise: causing the second robot arm to transition from operating in the calibration mode to operating in the instrument adjust mode using the more distal interface; and causing the second robot arm to transition from operating in the instrument adjust mode to operating in the surgical mode using the more distal interface.

The method may further comprise; causing the second robot arm to transition from operating in the surgical mode to operating in the instrument adjust mode.

The method may further comprise; causing the second robot arm to transition from operating in the surgical mode to operating in the instrument adjust mode using the less distal interface.

The second robot arm may be supported by a moveable arm support structure, and the method may further comprise: whilst the first robot arm is operating in the surgical mode and prior to inserting the second surgical instrument into the port, moving the arm support structure to a position adjacent to the patient.

Each of the first robot arm and second robot arm may further comprise an orientation interface, and the method may further comprise: after moving the moveable arm support structure supporting the second robot arm to a position adjacent to the patient, identifying a common direction by indicating a direction using the orientation interface of the first robot arm and indicating a corresponding direction using the orientation interface of the second robot arm.

In the surgical mode, the second robot arm may be remotely controlled by: receiving inputs relating to the second robot arm to the remote console; converting the inputs into control signals for the second robot arm in dependence on the determined fulcrum and the identified common direction; and controlling one or more joints of the series of joints of the second robot arm in dependence on the control signals so as to control the configuration of the second robot arm.

The surgical robotic system may comprise a third robot arm comprising a series of joints by which the configuration of that robot arm can be altered, the series of joints extending from a base at a proximal end of the robot arm to an attachment for a surgical instrument at a distal end of the robot arm, and the method may further comprise: whilst the first robot arm is operating in the surgical mode and prior to inserting the second surgical instrument into the port, retracting a third surgical instrument attached to the third robot arm from the patient's body.

The method may further comprise retracting the third surgical instrument from the patient's body by: enabling the configuration of the third robot arm to be altered in response to external forces, the freedom of motion of the third robot arm being limited such that the third surgical instrument can only move linearly in directions co-axial with the longitudinal axis of the third surgical instrument and away from the patient's body; and applying external forces to the third robot arm such that its configuration is altered in order to retract the third surgical instrument from the patient's body.

The third robot arm may further comprise one or more force sensors configured to sense forces at one or more joints of the series of joints of the third robot arm, and one or more motors configured to drive one or more joints of the series of joints of the third robot arm, and the method may further comprise: sensing, using the one or more force sensors, external forces at one or more joints of the series of joints of the third robot arm; resolving the sensed external forces so as to determine the components of the forces parallel with the longitudinal axis of the third surgical instrument and away from the patient's body; and driving, using the one or more motors, one or more joints of the series of joints of the third robot arm in dependence on the components of the forces parallel with the longitudinal axis of the third surgical instrument so as to alter the configuration of the third robot arm.

The method may further comprise: whilst the first robot arm is operating in the surgical mode and prior to inserting the second surgical instrument into the port, retracting the second surgical instrument from the patient's body; and performing a maintenance task on the second robot arm before inserting the second surgical instrument into the patient's body.

The method may further comprise retracting the second surgical instrument from the patient's body by: enabling the configuration of the second robot arm to be altered in response to external forces, the freedom of motion of the second robot arm being limited such that the second surgical instrument can only move linearly in directions parallel with the longitudinal axis of the second surgical instrument; and applying external forces to the second robot arm such that its configuration is altered in order to retract the second surgical instrument from the patient's body.

The second robot arm may further comprise one or more force sensors configured to sense forces at one or more joints of the series of joints of the second robot arm, and one or more motors configured to drive one or more joints of the series of joints of the second robot arm, and the method may further comprise: sensing, using the one or more force sensors, external forces at one or more joints of the series of joints of the second robot arm; resolving the sensed external forces so as to determine the components of the forces parallel with the longitudinal axis of the second surgical instrument; and driving, using the one or more motors, one or more joints of the series of joints of the second robot arm in dependence on the components of the forces parallel with the longitudinal axis of the second surgical instrument so as to alter the configuration of the second robot arm.

The surgical mode in which the first robot arm is operating may be an engaged surgical mode in which the configuration of the first robot arm and first surgical instrument is controlled by a remote surgeon console.

The surgical mode in which the first robot arm is operating may be a disengaged surgical mode in which the configuration of the first robot arm and first surgical instrument is controllable by a remote surgeon console.

According to a second aspect of the invention there is provided a control system of a surgical robotic system, the surgical robotic system comprising a first robot arm and a second robot arm, each of the first and second robot arms comprising a series of joints by which the configuration of that robot arm can be altered, the series of joints extending from a base at a proximal end of the robot arm to an attachment for a surgical instrument at a distal end of the robot arm, the control system being configured to reconfigure the surgical robotic system by: controlling the first robot arm to operate in a surgical mode in which a first surgical instrument attached to that first robot arm is inside a patient's body; and whilst the first robot arm is operating in the surgical mode: (i) controlling the second robot arm so as to permit a second surgical instrument attached to the second robot arm to be inserted into a port in the patient's body; (ii) determining a fulcrum about which the second surgical instrument pivots when the configuration of the second robot arm is altered whilst the second surgical instrument is inside the port; and (iii) controlling the second robot arm to operate in a surgical mode in which the configuration of the second robot arm and second surgical instrument is controlled in response to inputs received at a remote surgeon console whilst maintaining an intersection between the second surgical instrument and the determined fulcrum.

The fulcrum may be determined by: controlling the second robot arm so as to enable its configuration to be altered in response to external forces whilst the second surgical instrument is inside the port; and determining the fulcrum, the fulcrum being the point about which the surgical instrument of the second robot arm pivots whilst inside the port.

The second robot arm may further comprise one or more force sensors configured to sense external forces at one or more joints of the series of joints of the second robot arm, and one or more motors configured to drive one or more joints of the series of joints of the second robot arm, and the control system may be further configured to: control the one or more motors so as to drive one or more joints of the series of joints of the second robot arm in dependence on external forces sensed by the one or more force sensors so as to alter the configuration of the second robot arm.

The second robot arm may further comprise one or more position sensors configured to sense the position of one or more joints of the series of joints of the second robot arm and to record the position of one or more joints of the series of joints of the second robot arm at a plurality of instances whilst the configuration of the second robot arm is being altered, and the control system further may be configured to: determine, for each instance, a position of the distal end of the second robot arm in dependence on the respective recorded one or more joint positions; determine, for each instance, a vector of the second surgical instrument from the determined position of the distal end of the second robot arm in dependence on the respective recorded one or more joint positions; and determine the point of intersection of the determined vectors of the second surgical instrument so as to determine the fulcrum.

The control system may be further configured to: determine the fulcrum when controlling the second robot arm to operate in a calibration mode; and control the second robot arm to transition from operating in the calibration mode to operating in the surgical mode.

The second robot arm may further comprise a more distal interface and a less distal interface, and the control system may be further configured to: control the second robot arm to transition from operating in the calibration mode to operating in the surgical mode in response to an operator interaction with the more distal interface.

The control system may be further configured to: control the second robot arm so as to permit the second surgical instrument to be inserted into the port by controlling the second robot arm to operate in a compliant mode in which the configuration of the second robot arm can be altered in response to external forces; and control the second robot arm to transition from operating in the compliant mode to operating in the calibration mode.

The control system may be further configured to: control the second robot arm to transition from operating in the compliant mode to operating in the calibration mode in response to a user interaction with the more distal interface.

The control system may be further configured to: after determining the fulcrum, control the second robot arm to operate in an instrument adjust mode in which the configuration of the second robot arm can be altered in response to external forces but is constrained such that an intersection is maintained between the second surgical instrument and the determined fulcrum.

The control system may be further configured to: control the second robot arm to transition from operating in the calibration mode to operating in the instrument adjust mode; and control the second robot arm to transition from operating in the instrument adjust mode to operating in the surgical mode, and optionally control the second robot arm to transition from operating in the surgical mode to operating in the instrument adjust mode.

Each of the first robot arm and second robot arm may further comprise an orientation interface, and the control system may be further configured to: receive an input identifying a common direction in response to an operator indicating a direction using the orientation interface of the first robot arm and indicating a corresponding direction using the orientation interface of the second robot arm.

In the surgical mode, the second robot arm may be remotely controlled by the control system being configured to: receive inputs relating to the second robot arm to the remote console; convert the inputs into control signals for the second robot arm in dependence on the determined fulcrum and the identified common direction; and control one or more joints of the series of joints of the second robot arm in dependence on the control signals so as to control the configuration of the second robot arm.

The surgical robotic system may comprise a third robot arm comprising a series of joints by which the configuration of that robot arm can be altered, the series of joints extending from a base at a proximal end of the robot arm to an attachment for a surgical instrument at a distal end of the robot arm, and the control system may be further configured to: whilst controlling the first robot arm to operate in the surgical mode and prior to permitting the second surgical instrument to be inserted into the port, control the third robot arm so as to permit a third surgical instrument attached to the third robot arm to be retracted from the patient's body.

The control system may be further configured to permit the third surgical instrument to be retracted from the patient's body by: enabling the configuration of the third robot arm to be altered in response to external forces, the freedom of motion of the third robot arm being limited such that the third surgical instrument can only move linearly in directions co-axial with the longitudinal axis of the third surgical instrument and away from the patient's body.

The third robot arm may further comprise one or more force sensors configured to sense external forces at one or more joints of the series of joints of the third robot arm, and one or more motors configured to drive one or more joints of the series of joints of the third robot arm, and the control system may be further configured to: resolve external forces sensed by the one or more force sensors so as to determine the components of the forces parallel with the longitudinal axis of the third surgical instrument and away from the patient's body; and control the one or more motors so as to drive one or more joints of the series of joints of the third robot arm in dependence on the components of the forces parallel with the longitudinal axis of the third surgical instrument so as to alter the configuration of the third robot arm.

The control system may be further configured to, whilst controlling the first robot arm to operate in the surgical mode and prior to permitting the second surgical instrument to be inserted into the port, control the second robot arm so as to permit the second surgical instrument to be retracted from the patient's body; and control the second robot arm so as to permit the second surgical instrument to be inserted into the patient's body after a maintenance task has been performed on the second robot arm.

The control system may be further configured to permit the second surgical instrument to be retracted from the patient's body by: enabling the configuration of the second robot arm to be altered in response to external forces, the freedom of motion of the second robot arm being limited such that the second surgical instrument can only move linearly in directions parallel with the longitudinal axis of the second surgical instrument.

The second robot arm may further comprise one or more force sensors configured to sense external forces at one or more joints of the series of joints of the second robot arm, and one or more motors configured to drive one or more joints of the series of joints of the second robot arm, and the control system may be further configured to: resolve external forces sensed by the one or more force sensors so as to determine the components of the forces parallel with the longitudinal axis of the second surgical instrument; and control the one or more motors so as to drive one or more joints of the series of joints of the second robot arm in dependence on the components of the forces parallel with the longitudinal axis of the second surgical instrument so as to alter the configuration of the second robot arm.

The surgical mode in which the control system controls the first robot arm to operate may be: an engaged surgical mode in which the configuration of the first robot arm and first surgical instrument is controlled in response to inputs received at the remote surgeon console; or a disengaged surgical mode in which the configuration of the first robot arm and first surgical instrument is controllable in response to inputs received at the remote surgeon console.

### BRIEF DESCRIPTION OF THE DRAWINGS

The present invention will now be described by way of example with reference to the accompanying drawings. In the drawings:

FIG. 1 shows a typical surgical robotic system.

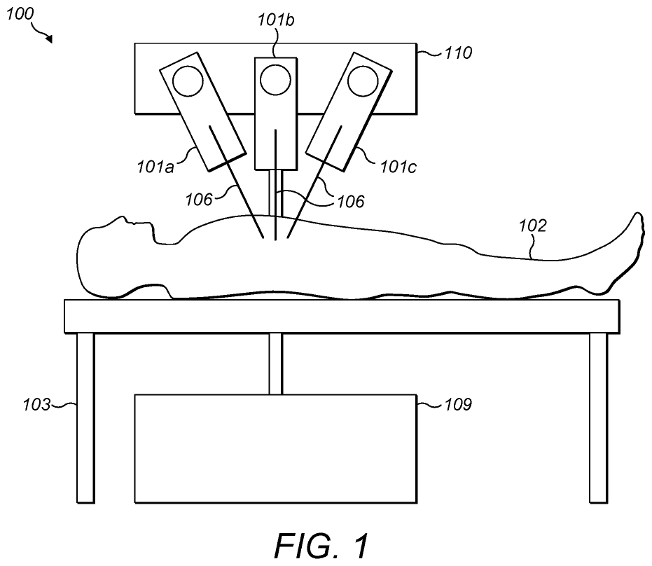

FIG. 2 shows a surgical robotic system.

FIG. 3 shows a surgical robot arm of a surgical robotic system.

FIG. 4 shows a start-up sequence of modes for a surgical robot arm.

FIG. 5 is a flow diagram showing the steps for calibrating a surgical robot arm.

FIG. 6 shows a plan view of a surgical robotic system.

FIG. 7 shows a flow diagram for reconfiguring a surgical robotic system in accordance with the principles described herein.

### DETAILED DESCRIPTION OF THE DRAWINGS

The following description is presented to enable any person skilled in the art to make and use the invention, and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown, but is to be accorded the widest scope consistent with the principles and features disclosed herein.

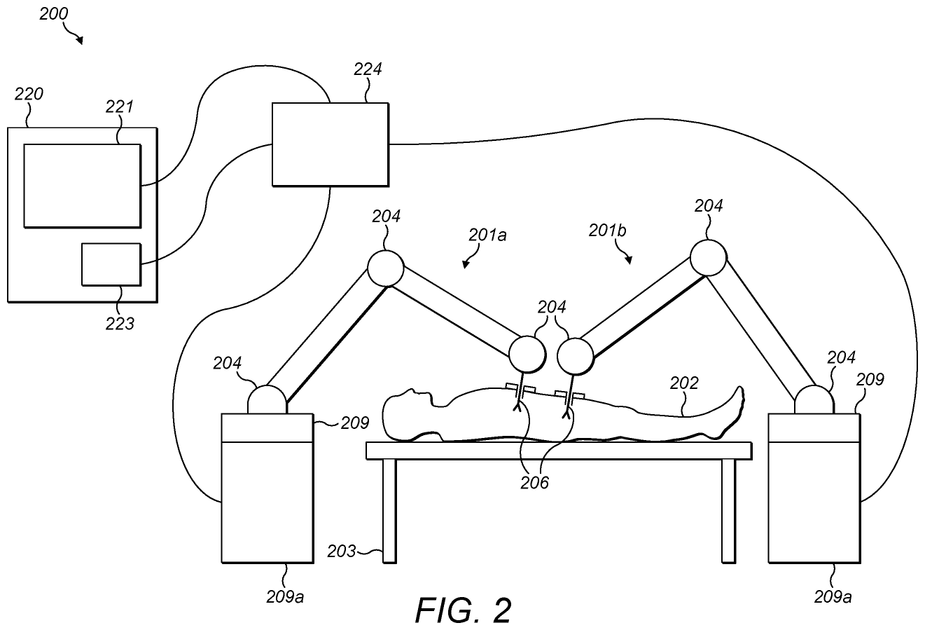

FIG. 2 shows a surgical robotic system. FIG. 2 shows a surgical robotic system 200 performing an invasive medical procedure on a patient 202. The patient 202 is positioned on an operating table 203. The surgical robotic system 100 comprises a first robot arm 201a and a second robot arm 201b. Although two robot arms 201a, 201b are shown in FIG. 2, it is to be understood that a surgical robotic system configured in accordance with the principles described herein may comprise any number of robot arms. Each robot arm 201a, 201b extends from a base 209 at its proximal end. Each robot arm 201a, 201b comprises a plurality of joints 204 by which the configuration of that robot arm can be altered.

Each robot arm 201a, 201b comprises an attachment for a surgical instrument 206 at its distal end. The surgical instrument may have a thin elongate shaft with an end effector at its distal end for performing aspects of the invasive procedure. The surgical instrument could, for example, be a cutting or grasping device, or an imaging device (such as an endoscope). Each surgical instrument 206 is insertable into a patient's body 202. Each surgical instrument 206 may be inserted into a patient's body 202 via a port. Each surgical instrument 206 may be inserted into the patient 202 through a different port.

The base 209 of each robot arm 201a, 201b is supported by a support structure 209a. The support structure 209a may be moveable. The support structure may be a cart or trolley. For example, each support structure may comprise one or more wheels (not shown) on which the support structure can be moved. In other examples, a moveable support structure may be mounted on one or more of: rails, sliders or bearings, or any other element on which the support structure can be moved. In an unshown example, the base 209a may be supported by a wall or ceiling mounted arm support structure (e.g. by being secured to that structure). Said wall or ceiling mounted arm support structure may be mounted on one or more rails, or any other suitable element, such that the wall or ceiling mounted arm support structure can be moved. An arm support structure may be moveably mounted on any other suitable surface (e.g. operating table 203). The moveable support structure may be equipped with brakes (not shown), such that the support structure can be caused to remain static when required (e.g. whilst the robot arm it supports is participating in an invasive medical procedure).

The configuration of each robot arm 201a, 201b may be remotely controlled in response to inputs received at a remote surgeon console 220. A surgeon may provide inputs to the remote console 220. The remote surgeon console comprises one or more surgeon input devices 223. For example, these may take the form of a hand controller and/or foot pedal. The surgeon console also comprises a display 221.

A control system 224 connects the surgeon console 220 to each surgical robot 201a, 201b. The control system receives inputs from the surgeon input device(s) and converts these to control signals to move the joints of the robot arms 104 and surgical instrument 206. The control system 224 sends these control signals to the robot, where the corresponding joints are driven accordingly. The control system 224 may be separate from the remote surgeon console 220 and the robot arms 201a, 201b. The control system 224 may be collocated with the remote surgeon console 220. The control system 224 may be collocated with one of the robot arms 201a, 201b. The control system 224 may be distributed between the remote surgeon console 220 and the robot arms 201a, 201b.

The configuration of each robot arm 201a, 201b may be controllable in response to external forces applied directly to that robot arm. For example, a member of the bedside team (e.g. an operating room nurse) may apply forces directly to a robot arm (e.g. by pushing a joint of the robot arm). This behaviour will be described in further detail herein.

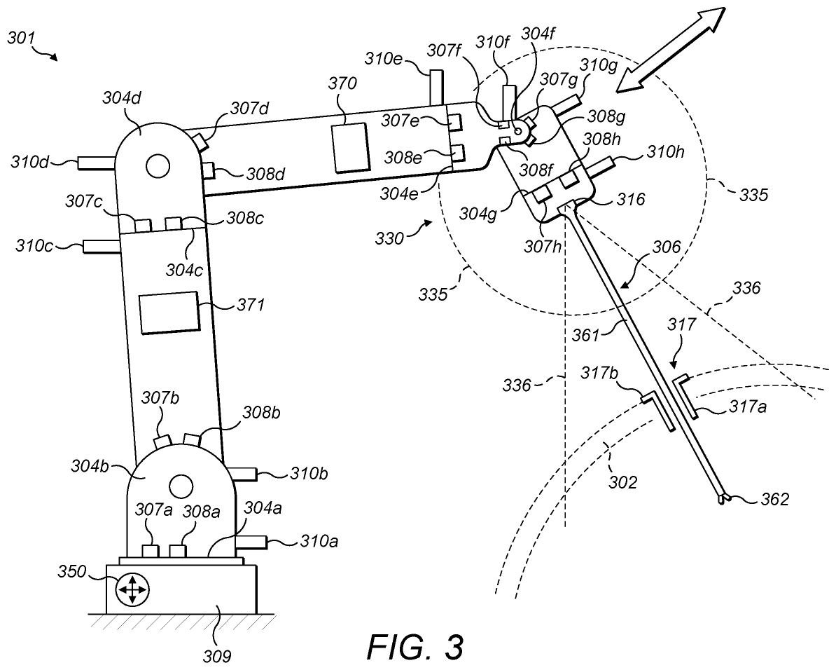

FIG. 3 shows an example of a robot arm 301. The robot arms 201a, 201b shown in FIG. 2 may have the same features as the robot arm 301 shown FIG. 3.

The robot arm comprises a base 309. The robot arm has a series of rigid arm members. Each arm member in the series is joined to the preceding arm member by a respective joint 304a-g. Joints 304a-e and 304g are revolute joints. Joint 304f is composed of two revolute joints whose axes are orthogonal to each other, as in a Hooke's or universal joint. Joint 304f may be termed a “wrist joint”. A robot arm could be jointed differently from the arm of FIG. 3. For example, joint 304d could be omitted and/or joint 304f could permit rotation about a single axis. The robot arm could include one or more joints that permit motion other than rotation between respective sides of the joint, such as a prismatic joint by which an instrument attachment can slide linearly with respect to more proximal parts of the robot arm.

The joints are configured such that the configuration of the robot arm can be altered allowing the distal end 330 of the robot arm to be moved to an arbitrary point in a three-dimensional working volume illustrated generally at 335. One way to achieve that is for the joints to have the arrangement illustrated in FIG. 3. Other combinations and configurations of joints could achieve a similar range of motion, at least within the zone 335. There could be more or fewer arm members.

The distal end of the robot arm 330 has an attachment 316 by means of which a surgical instrument 306 can be releasably attached. The surgical instrument has a linear rigid shaft 361 and an end effector 362 at the distal end of the shaft. The end effector 362 consists of a device for engaging in a procedure, for example a cutting, grasping or imaging device. As described herein, terminal joint 304g may be a revolute joint. The surgical instrument 306 and/or the attachment 316 may be configured so that the instrument extends linearly parallel with the rotation axis of the terminal joint 304g of the robot arm. In this example the instrument extends along an axis coincident with the rotation axis of joint 304g.

Joints 304e and 304f of the robot arm are configured so that with the distal end of the robot arm 330 held at an arbitrary location in the working volume 335 the surgical instrument 306 can be directed in an arbitrary direction within a cone. Such a cone is illustrated generally at 336. One way to achieve that is for the terminal part of the arm to comprise the pair of joints 304e and 304f whose axes are mutually arranged as described above. Other mechanisms can achieve a similar result. For example, joint 304g could influence the attitude of the instrument if the instrument extends In a direction which is not parallel to the axis of joint 304g.

The surgical instrument 306 may be inserted into the patient's body through a port 317. The port 317 may comprise a hollow tube 317a. The hollow tube 317a may pass through the outer tissues 302 of the patient so as to limit disruption to those tissues as the surgical instrument is inserted and removed, and as the instrument is manipulated within the patient's body. The port 317 may comprise a collar 317b. The collar 317b may prevent the port 317 being inserted too far through the outer tissues 302 of the patient. In other examples (not shown), the surgical instrument may be inserted directly into the patient's body, e.g. through a natural orifice such as the throat. A natural orifice through which the surgical instrument is inserted into the patient's body is also referred to herein as a “port”.

The robot arm 301 comprises a series of motors 310a-h. With the exception of the compound joint 304f, which is served by two motors, each motor is arranged to drive rotation about a respective joint of the robot arm. The motors are controlled by a control system (such as control system 224 shown in FIG. 2). The control unit comprises a processor and a memory. The memory stores, in a non-transient way, software code that can be executed by the processor to cause the processor to control the motors 310a-h in the manner described herein.

The robot arm 301 may comprise a series of sensors 307a-h and 308a-h. These sensors may comprise, for each joint, a position sensor 307a-h for sensing the rotational position of the joint and a force sensor 308a-h for sensing forces (such as torque) applied about the joint's rotation axis. Compound joint 304f may have two pairs of sensors. One or both of the position and force sensors for a joint may be integrated with the motor for that joint. The outputs of the sensors are passed to the control system where they form inputs for the processor.

The robot arm 301 may also comprise an orientation interface 350. The orientation interface 350 may be, for example, a button or set of buttons accessible by a member of the bedside team (e.g. an operating room nurse). Orientation interface 350 can be used to identify a common direction for the robot arms of a surgical robotic system (such as surgical robotic system 200 shown in FIG. 2). The output of the orientation sensor may be passed to the control system where it forms an input to the processor. The orientation interface 350 will be described in further detail below.

The robot arm 301 may also comprise one or more interfaces 370, 371. Interfaces 370, 371 may be, for example, a button or set of buttons accessible by a member of the bedside team (e.g. an operating room nurse). Interfaces 370, 371 can be used to select between the operating modes of a robot arm. Interfaces 370, 371 will be described in further detail below.

Configuring a Robot Arm

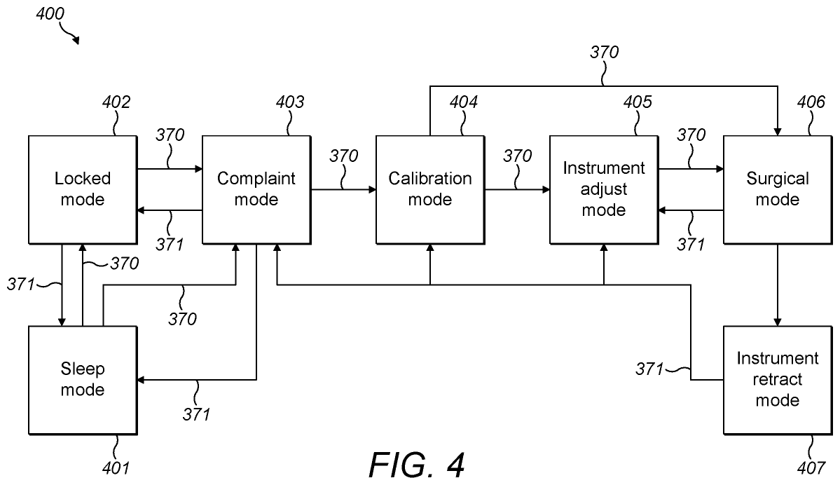

In order to configure a robot arm such that it can be used as part of a surgical robotic system, a start-up sequence of modes may be used. FIG. 4 shows a start-up sequence of modes for a surgical robot arm. It is to be understood that the control system of the surgical robotic system (such as control system 224 shown in FIG. 2) controls the operation of each robot arm in the surgical robotic system—i.e. in accordance with the various operating modes described with reference to FIG. 4. It is to be understood that the control system controls the operation and behaviour of each robot arm in each operating mode, and controls the transitioning of each robot arm between operating modes as described herein.

As shown in FIG. 4, a robot arm may be operated in a sleep mode 401, followed by a locked mode 402, followed by a compliant mode 403, followed by a calibration mode 404, followed by an adjust mode 405, followed by a surgical mode 406. That said, it is not necessary for a robot arm to be operated in all of the modes shown in FIG. 4 in order to be configured such that it can be operated in a surgical mode 406. For example, a robot arm may be operated in the calibration mode 404, directly followed by the surgical mode 406. A robot arm may be sequentially operated in any combination of the modes shown in FIG. 4, by performing any sequence mode transitions shown to be possible by the arrows in FIG. 4. For example, a robot arm may be operated in the calibration mode 404, directly followed by the surgical mode 406, followed by the instrument adjust mode 405, followed by returning to the surgical mode 406.

The robot arm may be operable in a sleep mode 401. In the sleep mode 401, the robot arm may adopt a configuration suitable for storing or transporting the robot arm. Such a configuration may be a compact configuration. For example, in said configuration the proximal arm members may be substantially parallel to one another—although other compact configurations are also suitable. A surgical instrument 306 may not be attached to the robot arm when it is operating in the sleep mode 401.

In the sleep mode 401 the robot arm may resist external forces so as to maintain its configuration. Brakes may be applied at one or more of the joints 304a-g of the robot arm such that the robot arm can resist external forces so as to maintain its configuration. Said brakes may be of any suitable type, such as electronic, magnetic or mechanical brakes. In the sleep mode 401, the robot arm does not change its configuration in response to inputs at the remote surgeon console (e.g. remote surgeon console 220 shown in FIG. 2).

The robot arm may be operable in a locked mode 402. In the locked mode, the robot arm may adopt a “horse-shoe” configuration (e.g. of the type shown in FIGS. 2 and 3). The “horse-shoe” configuration is preferable in the locked mode 402 as the orientation of the joints of the robot arm are away from their joint limits and singular configurations. That is, the “horse-shoe” configuration is such that a large range of movement of the robot arm is possible from that configuration. In the “horse-shoe” the robot arm can be placed near the port in a configuration which is similar to the final/optimal configuration that will be used when performing surgery—so that the operating staff have a good idea on how the surgical setup will look like. On transitioning from the sleep mode 401 to the locked mode 402, the configuration of the robot arm may be altered from a compact configuration to the “horse-shoe” configuration. Such a change may be driven by one or more of the series of motors 310a-h.

A surgical instrument 306 may be attached to the robot arm when it is operating in the locked mode 402. In the locked mode 402 the robot arm may resist external forces so as to maintain its configuration. Brakes may be applied at one or more of the joints 304a-g of the robot arm such that the robot arm can resist external forces so as to maintain its configuration. Said brakes may be of any suitable type, such as electronic, magnetic or mechanical brakes. In the locked mode 402, the robot arm does not change its configuration in response to inputs at the remote surgeon console (e.g. remote surgeon console 220 shown in FIG. 2).

A surgical instrument 306 may be attached to the robot arm when it is operating in the locked mode 402. A drape (not shown) may be applied to the robot arm when it is operating in the locked mode 402.

The operating mode of the robot arm may also be transitioned from the locked mode 402 to the sleep mode 401, such as when the robot arm is being prepared for storage after a procedure has been completed.

The robot arm may be operable in a compliant mode 403. As shown in FIG. 4, the operating mode of the robot arm may be caused to transition from the locked mode 402 to the compliant mode 403. The operating mode of the robot arm may also be caused to transition directly from the sleep mode 401 to the compliant mode 403. On transitioning from the sleep mode 401 to the compliant mode 403, the configuration of the robot arm may be changed from a compact configuration to the “horse-shoe” configuration.

In the compliant mode 403, the configuration of the robot arm is changeable in response to external forces applied directly to that robot arm. For example, a member of the bedside team (e.g. an operating room nurse) may apply forces directly to a robot arm (e.g. by pushing a joint of the robot arm). In the compliant mode 403 the control system (such as control system 224 shown in FIG. 2) controls the robot arm to maintain a position in which it is placed by means of external forces applied directly to the robot arm.

To achieve this, the control system receives inputs from the position and force sensors 307a-h and 308a-h. From the position sensors the control system can determine the current configuration of the robot arm. The control system stores for each element of the robot arm, and the surgical instrument, its mass, the distance of its centre of mass from the preceding joint of the robot arm and the relationship between the centre of mass and the positional output of the position sensor for the preceding joint. The current configuration of the robot arm could be inferred by other means. For example, camera-based positioning systems may be used to track points in space, such as fiducial markers attached to the robot arm. This technique could be used to determine the joint angles. Other techniques include inferring the position of a joint using a current sensors. For example, the position of a joint can be inferred from the amount of current passing through the motor and assuming a given relationship to be constant.

Using that information, the control system models the effect of gravity on the components of the robot arm for the current configuration of the robot arm and estimates a force (e.g. a torque) due to gravity on each joint of the robot arm. The processor then drives the motor 310a-h of each joint to apply a force (e.g. a torque) that will exactly oppose the calculated gravitational force. With this control strategy an operator (e.g. an operating room nurse) can push or pull any part of the robot arm to a desired position, and the part will stay in that position notwithstanding the effect of gravity on it and on any parts depending from it. A force on the arm may result in a torque about multiple joints. The control system can be programmed to decide to prioritise certain ones of the joints for neutralising the torque. In examples, some joints could be locked in position and others could move compliantly, the position of a given link or point in space could be prioritized rather than sets of joints.

Each motor 310a-h may be controlled in response to the force (e.g. torque) measured about the respective joint. When the measured force at a joint is adjusted for gravity, any remaining sensed force represents a force applied by an external force (e.g. due to a push or pull on the robot arm). In response to that force the control system may control the respective motor 310a-h so as to alter the configuration of the robot arm. For example, this may be achieved by controlling the motors 310a-h to move their respective joints 304a-g in a direction so as to reduce the measured force, and at a rate dependant on the magnitude of the measured force. In this way, the member of the bedside staff may feel that that the robot arm is moving freely in response to the force they are applying—when in fact it is the motors of the robot arm driving the movement.

The compliant mode 403 can be used to insert the surgical instrument into a port in the patient's body. That is, the compliant mode 403 may be used to insert an end effector 362 of the surgical instrument into a port 317 positioned in the patient's body. In the compliant mode 403, the surgical instrument may be positioned at the entrance of the port 317, such that the end effector 362 is concentric with the entrance of the port 317. The entrance of the port may protrude slightly from the patient's body. The end effector 362 of the surgical instrument can of course be inserted further into the port 317 in the compliant mode 403. As described herein, the port may alternatively be a natural orifice in the patient's body, such as the throat.

Referring again to FIG. 3, with the robot arm in the compliant mode 403, an operator (e.g. an operating room nurse) can grasp one or both of the robot arm 301 and the surgical instrument 306. The operator can then apply external forces so as to alter the configuration of the robot arm 301 such that the elongate axis of the shaft 361 of the instrument is roughly aligned with the passageway through the hollow tube 317a of the port 317. The operator can then apply an external force (e.g. push) to the robot arm and/or the instrument such that the instrument moves roughly parallel to its elongate axis and passes into the passageway in the port 317.

The operating mode of the robot arm may also be transitioned from the compliant mode 403 to the locked mode 402 or the sleep mode 401, such as when the robot arm is being prepared for storage after a procedure has been completed.

The robot arm may be operable in a calibration mode 404. As shown in FIG. 4, the operating mode of the robot arm is caused to transition from the compliant mode 403 to the calibration mode 404.

As described herein, the surgical instrument 306 can be inserted into the patient's body, e.g. via port 317. This can be performed in the compliant mode 403 or the calibration mode 404. This is because the robot arm can respond to external forces in the calibration mode 404 in the same manner as in the compliant mode 403.

In the calibration mode 404 the location of port 317 relative to the robot arm 301 is estimated. For example, the location of port 317 may be estimated relative to the wrist joints 304e-g. Specifically, a fulcrum may be determined, the fulcrum being a point about which the surgical instrument 306 pivots when inside in the patient's body. The fulcrum may be the natural rotation centre of the port 317.

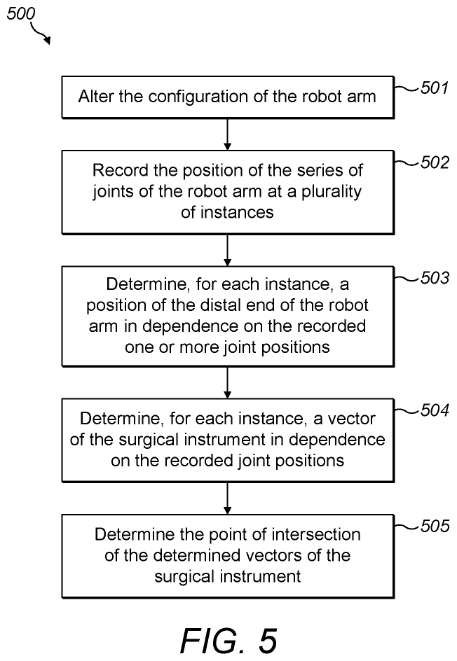

The control system (e.g. such as control system 224 shown in FIG. 2) of the robot arm 301 may be capable of determining the fulcrum by means of a calibration process that is performed whilst the surgical instrument 306 is inside the port 317. FIG. 5 is a flow diagram showing the steps of such a calibration process.

Whilst the surgical instrument is inside the port 317, the configuration of the robot arm is altered 501. The configuration of the robot arm 301 is altered by the application of external forces directly onto the robot arm. The robot arm can be moved generally transversely to the shaft 361 of the instrument 306. This causes the port 317 to apply a lateral force on the instrument shaft 316. That force is accommodated by motion about the joint 304f. As the configuration of the robot arm is being altered, the position sensors 307a-h record the position of each joint of the robot arm. The position sensors 307a-h record 502 the positions of each joint of the robot arm at a plurality of instances. That is, the position sensors 307a-h record the positions of each joint of the robot arm at a plurality of points in time. Position information may be recorded irregularly or at predetermined intervals, e.g. every 0.5 seconds. The position sensors provide the recorded position information to the control system. The control system uses this received information to determine: (a) the position of the distal end of the robot arm relative to the base and (b) the vector of the instrument shaft 361 relative to the distal end of the robot arm. Position (a) and vector (b) may be termed a data pair. The control system may determine a data pair for each instance at which the position sensors recorded position information. That is, for each instance, the control system determines a position of the distal end of the robot arm 503 in dependence on the recorded one or more joint positions. In addition, for each instance, the control system determines a vector of the surgical instrument from the determined position of the distal end of the second robot arm 504 in dependence on the recorded joint positions. Since the axis of the instrument shaft 361 passes through the passageway of the port 317, the passageway of the port lies along that vector. As the distal end of the robot arm is moved, the control system calculates multiple pairs of distal end positions and instrument shaft vectors. Those vectors all converge, from their respective distal end position, on the natural rotation centre of the passageway of the port 317. By collecting a series of those data pairs and then solving for the mean location where the instrument shaft vectors converge the control system estimates the location of the port relative to the robot arm. That is, the control system determines the point of intersection of the determined vectors of the surgical instrument 505 so as to determine the fulcrum. The control system then stores the determined fulcrum in non-transient form in memory for later use.

During step 501, the configuration of the robot arm may be altered such that the distal end of the robot arm is moved in two dimensions: e.g. with (i) components parallel to a direction that is transverse to the instrument shaft 361 and also with (ii) components orthogonal to that direction but transverse to the instrument shaft 361. To do this, the operator (e.g. a member of the bedside team) may gyrate the distal end of the robot arm about a point generally aligned with the natural axis of the hollow tube 317a of the port.

The number of data pairs determined in step 502, and used in steps 503 to 505, to determine the fulcrum with acceptable precision depends on factors such as the accuracy of the robot arm's position sensors and the extent to which the operator moves the arm laterally during the calibration process. The control system may determine that the fulcrum has been estimated adequately once sufficient coherent measurements have been gathered such that the variance between estimates of the fulcrum derived using successive measurements has reduced below a predefined level.

After determining the fulcrum, the robot arm may be operable in an instrument adjust mode 405. As shown in FIG. 4, the operating mode of the robot arm may be caused to transition from the calibration mode 404 to the instrument adjust mode 405. The operating mode of the robot arm may be caused to transition from the surgical mode 406 to the instrument adjust mode 405.

In the instrument adjust mode 405, the configuration of the robot arm can be altered in response to external forces in the same manner as in the compliant mode 403, with the exception that the configuration of the robot arm is constrained such that an intersection is maintained between the instrument shaft 361 and the determined fulcrum. The instrument adjust mode 405 can be used to adjust the position of the instrument within the patient's body. For example, instrument adjust mode 405 can be used to position the instrument in an optimal position to begin a procedure. For example, as described herein, the in the compliant mode 403, the surgical instrument may be positioned at the entrance of the port 317, such that the end effector 362 is concentric with the entrance of the port 317. The instrument adjust mode 405 may be used to assist insertion of the instrument further into the patient's body (e.g. towards an intended surgical site). In this example, the control system (such as control system 224 in FIG. 2) uses the determined position of the fulcrum to control the arm to adopt a configuration in which the instrument is generally aligned with the port passage. Then, an operator (e.g. a member of the bedside team, such as an operating room nurse) can insert the instrument further through the port by applying external forces to the robot arm. Whilst the instrument is being inserted further through the port, the control system controls the robot arm such that the shaft of the instrument intersects the fulcrum. That is, an intersection between the instrument and the fulcrum is maintained.

After determining the fulcrum, the robot arm is operable in a surgical mode 406. As shown in FIG. 4, the operating mode of the robot arm may be caused to transition directly from the calibration mode 404 to the surgical mode 406, or from the instrument adjust mode 405 to the surgical mode 406.

In the surgical mode 406, the configuration of the robot arm may be remotely controlled in response to inputs received at a remote surgeon console (such as remote surgeon console 220 shown in FIG. 2). A surgeon may provide inputs to the remote console 220. The remote surgeon console comprises one or more surgeon input devices 223. For example, these may take the form of a hand controller and/or foot pedal.

In the surgical mode 406 the operator (e.g. a surgeon) uses the remote surgeon console to signal a desired position of the end effector 362. The control system (such as control system 224 shown in FIG. 2) determines a configuration of the joints of the robot arm that will result in the end effector 362 being placed in that position. There may be multiple configurations of the robot arm that will result in the end effector 362 being placed in the desired position. The control system may select between those configurations based on an algorithm that seeks to avoid collisions between the robot arm and other objects known to the control system to be close to the robot arm, or that seeks to minimise the amount of movement of the joints to reach the new configuration. Once the control system has selected a new configuration it signals the joints 304a-g to adopt the states required to bring the arm into that configuration. In this way, in the surgical mode 406 the operator (e.g. a surgeon) can signal the end effector 362 to move to a desired location.

The control system uses the determined fulcrum to assist in controlling the configuration of the robot arm when the robot arm is operating in the surgical mode 406. The control system is configured, e.g. by means of the software stored in memory, to select a configuration of the arm for which both (i) the end effector 362 is at the desired position and (ii) the shaft 361 of the instrument 306 passes through the determined fulcrum, and to move the arm to that configuration. In that way the end effector 362 can be provided at the desired position with relatively little disruption to the outer tissues of the patient.

The surgical mode 406 may comprise an engaged surgical mode and a disengaged surgical mode. In the engaged surgical mode, the configuration of the robot arm and the surgical instrument is controlled by a remote surgeon console as described herein. In the disengaged surgical mode, the configuration of the robot arm and surgical instrument is controllable by a remote surgeon console. That is, in the disengaged surgical mode the fulcrum about which the surgical instrument pivots when the configuration of the robot arm is altered is known—and thus it is possible for the configuration of the robot arm and the surgical instrument to be controlled by a remote surgeon console. However, in the disengaged surgical mode, the configuration of the surgical instrument may temporarily be locked or maintained. For example, a surgeon may opt to place a robot arm in the disengaged surgical mode in order to take a rest, or such that they can focus their attention of the control of a different robot arm (e.g. during a particularly difficult part of a procedure). The surgeon may control the transition between the engaged and disengaged surgical modes—e.g. via an interface on the remote surgeon console, or by instructing a member of the operating room staff to interact with an interface on the robot arm itself.

When operating in the surgical mode 406, certain portions of the robot arm may exhibit compliant-like behaviour. For example, the configuration of the elbow joint 304d may be capable of being altered in response to external forces in the manner described herein, so long as the configuration of the instrument 306 is not affected. Enabling such compliant-like behaviour whilst the robot arm is operating in the surgical mode allows, for example, an operator of the robotic surgical system to move the elbow of the robot arm (e.g. so that they can access the patient during the procedure). In order to implement such compliant-like behaviour the control system may define an allowed area or volume for one or more parts the robot (e.g. the set of wrist joints 304e-g), such that the movement of those parts in response to externally applied forces is confined within that allowed area or volume. The allowed area or volume is defined such that movements within that area or volume in response to externally applied forces do not cause the configuration of the instrument 306 to be affected.

In examples where the robot arm comprises an orientation interface, the control system may also use the identified common direction to translate inputs at the remote surgeon console into suitable control signals for controlling the configuration of the robot arm when operating in the surgical mode 406.

The robot arm may also be operable in an instrument retract mode 406. As shown in FIG. 4, the operating mode of the robot arm may be caused to transition from the surgical mode 406 to the instrument retract mode 407.

The instrument retract mode 407 is engaged in order to remove the instrument 306 from a patient's body (e.g. at the end of a procedure). In the instrument retract mode 407, the control system controls the motors 310a-h to reconfigure the robot arm so as to cause the instrument 306 to be retracted from the port along the longitudinal axis of the instrument. The longitudinal axis of the instrument may be co-axial with the instrument shaft 361. The control system can achieve this by controlling the motors 310a-h so as to permit the robot arm to be reconfigured by the action of external forces applied to the robot arm, similar to the compliant mode 403. That is, in the instrument retract mode 407 the control system enables the configuration of the robot arm to be altered in response to external forces, but limits the freedom of motion of the robot arm such that the surgical instrument can only move linearly in directions parallel and/or co-axial with its longitudinal axis and away from the patient's body. In other words, an external force applied to the robot arm parallel with the shaft 361 of the instrument 306 and in a direction away from the patient's body, causes the instrument to be extracted from the patient's body. On detecting external force applied in this direction, the control system responds signalling the motors 310a-h of the appropriate joints to drive the joints to move in the direction that the external force is applied. The force of gravity on each joint is opposed as described above with respect to the compliant mode 403. In this way, an operator (e.g. a member of the bedside team, such as an operating room nurse) can manually push or pull the robot arm in a direction away from the patient's body, and the robot arm will respond by moving in that direction. Thus, the robot arm provides the sensation to the operator of moving freely under their push or pull to withdraw the instrument from the patient's body.

The control system detects external forces applied to the robot arm by means of force sensors 307a-h. The control system uses this sensor input to determine if an applied external force is acting along and/or parallel to the longitudinal axis of the instrument and away from the patient's body. In examples where the instrument extends along an axis coincident with the rotation axis of joint 304g, the control system may use the sensor input to determine if an applied external force is coincident (and therefore also inherently parallel) with the longitudinal axis of the instrument and away from the patient's body. In examples where the instrument extends linearly parallel with the rotation axis of the terminal joint 304g of the robot arm (but not necessarily co-axial with that rotation axis), the control system may use the sensor input to determine if an applied external force is parallel with the longitudinal axis of the instrument and away from the patient's body.

In order to make this determination, the control system resolves the detected applied forces, and signals the motors 310a-h to drive the robot arm based only on the components of the forces parallel with the longitudinal axis of the instrument. In the instrument retract mode 407, components of applied external forces in a direction transverse to the longitudinal axis of the instrument are not acted upon. External forces applied in such directions are thereby resisted. Further, components of applied external forces acting along the longitudinal axis of the instrument towards the patient's body are not acted upon. External forces applied in this direction are thereby resisted.

The operating mode of the robot arm may be transitioned from the instrument retract mode 407 to any of the complaint mode 403, the calibration mode 404, or the instrument adjust mode 405.

As described herein with reference to FIG. 3, robot arm 301 may comprise one or more interfaces 370, 371. Interfaces 370, 371 may be, for example, a button or set of buttons accessible by a member of the bedside team (e.g. an operating room nurse). Interfaces 370, 371 can be actuated to transition between the operating modes described with reference to FIG. 4.

Referring again to FIG. 3, interface 370 may be positioned in a more distal position than interface 371. That is, interface 370 may be positioned closer to the surgical instrument 306 than interface 371. For example, interface 370 may be positioned on or near to the wrist joints 304e-g, whilst interface 371 is positioned on or near elbow joints 304d. The robot arm 301 may be configured such that an operator interaction with the more distal interface 370 causes the operating mode of the robot arm to transition “towards” the surgical mode 406. The operating mode transitions considered to be “towards” the surgical mode 406 are labelled 370 in FIG. 4. For example, transitions “towards” the surgical mode 406 include: transitions from the sleep mode 401 to the locked mode 401 or the compliant mode 403; a transition from the locked mode 402 to the compliant mode 403; a transition from the compliant mode 403 to the calibration mode 404; and transitions from the calibration mode 404 to the instrument adjust mode 405 or the surgical mode 406. The robot arm 301 may be configured such that an operator interaction with the less distal interface 371 causes the operating mode of the robot arm to transition “away from” the surgical mode 406. The operating mode transitions considered to be “away from” the surgical mode 406 are labelled 371 in FIG. 4. For example, transitions “away from” the surgical mode 406 include: a transition from the surgical mode 406 to the instrument adjust mode 405; transitions from the instrument retract mode 407 to any of the compliant mode 403, calibration mode 404 or instrument adjust mode 405; transitions from the compliant mode 403 to the locked mode 402 or the sleep mode 401; and a transition from the locked mode 402 to the sleep mode 401. For example, if a robot arm were to be operating in the compliant mode 403, an interaction with: (i) the more distal interface 370 may cause the operating mode to transition to the calibration mode 404, and (ii) the less distal interface 371 may cause the operating mode to transition to the locked mode 402. In this manner, the selection of the next operating mode is more intuitive to the operator.

One or more conditions may be associated with each operating mode. These conditions may determine whether that mode can be accessed. For example, the surgical mode may require that the fulcrum has been determined and that the instrument is inserted by at least a predetermined distance within the patient's body (e.g. to ensure that the instrument is observable by the surgeon through an endoscope within the patient's body). Under certain conditions (e.g. under safety alarm situations) some modes may not be available.

Re-Configuring a Surgical Robotic System

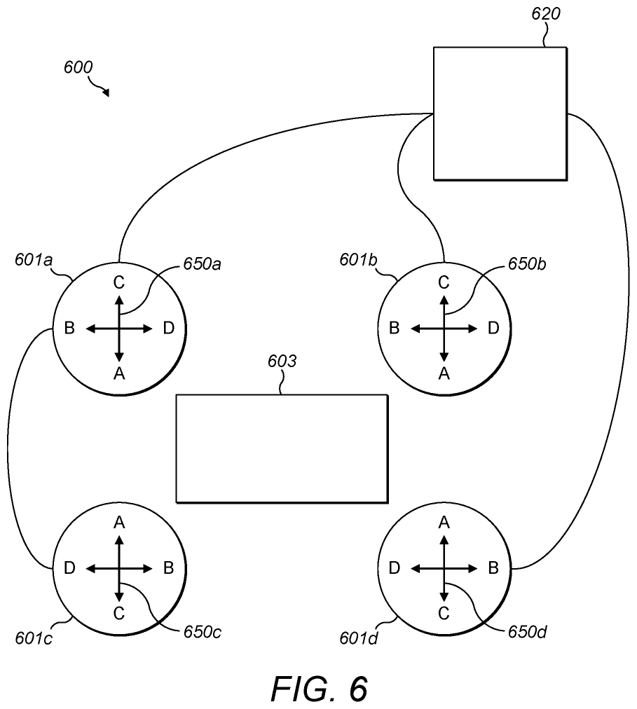

The re-configuration of a surgical robotic system will be described with reference to FIG. 6. FIG. 6 shows a plan view of a surgical robotic system. Surgical robotic system 600 comprises robot arms 601a, 601b, 601c, 601d. For simplicity, the robot arm linkages and joints of each of robot arms 601a-d are not shown in FIG. 6. Each robot arm 601a-d may comprise equivalent features to robot arm 301 described with reference to FIG. 3. Although four robot arms 601a-d are shown in FIG. 6, it is to be understood that a surgical robotic system configured in accordance with the principles described herein may comprise any number of robot arms.

Robot arms 601a-d are positioned about operating table 603. For simplicity, no patient is shown in FIG. 6. FIG. 6 shows a robot arm 601a-d positioned on each corner of operating table 603, but is to be understood that robot arms 601a-d may be positioned in any other arrangement.

FIG. 6 also shows remote surgeon console 620. Control system 624 is not shown in FIG. 6—but could be located in any of the positions described previously herein. It is to be understood that the control system of the surgical robotic system is configured to reconfigure the surgical robotic system by controlling the operation of each robot arm in the surgical robotic system—i.e. in accordance with the various operating modes described with reference to FIG. 4. Robot arms 601a-d may be connected to console 620 by wired, or wireless, connections. Said connections may provide control signals, and optionally power, from the console 620 to each robot arm 601a-d. One or more of the robot arms 601a-d may be directly connected to a power source (i.e. not connected to a power source via the console) and/or may be powered by a local power source, such as a battery. Said connections may also provide feedback from the robot arm 601a-d to the console 620. A suitable wired connection for communicating control signals is an ethernet field bus.

As shown in FIG. 6, robot arms 601 b and 601d are connected to console 620 by direct connections. Robot arm 601a is also connected to console 620 by a direct connection. Robot arm 601c is not connected to console 620 by a wired connection, but rather is connected to robot arm 601a which itself is connected to console 620 by a direct connection. Robot arms 601a and 601c and 130 may be considered to be part of a “daisy chain”. All robot arms in a surgical robotic system may be connected to a console by direct connections, or all robot arms may be part of a “daisy chain”, or any mixture of direct connections and “daisy chain” connections may be used (e.g. as shown in FIG. 6).

At any point in time, each robot arm 601a-d in surgical robotic system 600 may be operating in a different one of the modes shown in FIG. 5. At any point in time, each robot arm in a surgical robotic system may be operating in the same mode.

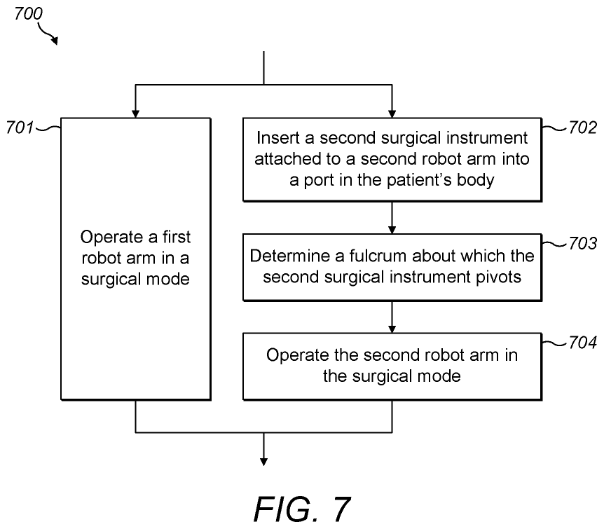

FIG. 7 shows a flow diagram for reconfiguring a surgical robotic system in accordance with the principles described herein. A robot arm may be added to a surgical robotic system whilst a robot arm already part of the surgical robotic system is operating in the surgical mode 406. For example, with reference to FIG. 6, a procedure may be ongoing using only first robot arm 601a. That is, the surgical instruments of robot arms 601b-d may not initially be inserted into the patient's body. The first robot arm 601a may be operating in the surgical mode 406. The second robot arm 601b may be added to surgical robotic system 600 whilst the first robot arm 601a is operating in a surgical mode 406. Adding a second robot arm 601b to a surgical robotic system involves inserting 702 the surgical instrument it carries (“the second surgical instrument”) into a port in the patient's body and configuring that robot arm such that it can be operated in a surgical mode 406. That is, the fulcrum about which the second surgical instrument pivots whilst inside the port is determined 703 (as described herein with reference to the calibration mode 404). The second robot arm 601b can then be operated 704 in the surgical mode in which the configuration of the second robot arm and the second surgical instrument is controlled by the remote surgeon console 620 whilst maintaining an intersection between the second surgical instrument and the determined fulcrum (as described herein with reference to the surgical mode 406). The first robot arm 601a may be continually operated in the surgical mode whilst the second robot arm 601b is being added to surgical robotic system 600. That is, the second robot arm 601b may be added to surgical robotic system 600 without interrupting the operation of the first robot arm 601a in the surgical mode 406. In other words, the operation 701 of the first robot arm 601a in the surgical mode 406 and the addition 702, 703, 704 of the second robot arm 601b to the surgical robotic system 600 may be performed concurrently. The first robot arm 601a may be operated 701 in the engaged surgical mode or the disengaged surgical mode, or sequentially in the engaged and disengaged surgical modes, as described herein whilst the second robot arm 601b is being added 702, 703, 704 to the surgical robotic system 600. Adding the second robot arm 601b to the surgical robotic system may involve operating the second robot arm 601b in any one of more of the sleep mode 401, locked mode 402, compliant mode 403, calibration mode 404 and instrument adjust mode 405 as described with reference to FIG. 4, prior to operating the second robot arm 601b in the surgical mode 406.

As described herein, the robot arm may be supported by a moveable support structure. Prior to adding a robot arm to a surgical robotic system, the robot arm may be moved into a position adjacent to the patient. A robot arm may be moved from a position remote from the patient into a position adjacent to the patient. For example, a spare robot arm may be provided in an operating room, or external to the operating room, for use when required (e.g. for any of the reasons previously given herein). Alternatively, a robot arm may be moved from a position adjacent to the patient into a different position adjacent to the patient (e.g. for the reasons given in the preceding paragraph). After a robot arm has been moved into a position adjacent to the patient, it may be added to the surgical robotic system. The robot arm could be moved into a position adjacent to the patient in any of the sleep mode 401, locked mode 402 or compliant mode 403.

As described herein with reference to FIG. 3, each of robot arms 601a-d may comprise an orientation interface 650a-d. The orientation interface 650a-d of each robot arm 601a-d may be, for example, a button or set of buttons accessible by a member of the bedside team (e.g. an operating room nurse). Although the orientation interfaces 601a-d shown in FIG. 6 are shown indicating four directions, it is to be understood that any number of directions may be indicated.

The orientation interface 650a-d of each robot arm 601a-d can be used to identify a common direction for the robot arms 601a-d of surgical robotic system 600. In an example, a direction may be selected using each orientation interface 650a-d such that all of the selected directions point in the same direction. For example, with reference to FIG. 6, direction A may be selected for robot arms 601a and 601b, and direction C may be selected for robot arms 601c and 601d.

The common direction may be used by the robotic surgical system 600 in order to translate inputs to the remote console 620 into suitable control signals for one or more of the robot arms 601a-d, when those robot arms are operating in the surgical mode 406. The common direction may be identified in any of the sleep mode 401, locked mode 402 or compliant mode 403.

The robot arms may not have orientation interfaces. For example, in some surgical robotic systems the robot arms are positioned in predetermined locations and orientations. For example, a jig may be provided in the operating room which enables each robot arm to be accurately positioned in a predetermined location and orientation, a camera based tracking system may be provided for detecting the relative location and orientation of the robot arms, or locating devices may be within the robot arms for detecting the relative location and orientation of the robot arms.

A robot arm may need not be moved prior to being added to a surgical robotic system. That is, a robot arm may already be located in a position adjacent to the patient. For example, with reference to FIG. 6, robot arms 601a-c may be part of a surgical robotic system. Robot arm 601d may be located in the position shown in FIG. 6, but may not be being used as part of the surgical robotic system. That is, robot arm 601d may be a redundant robot arm. If required during a procedure, robot arm 601d could be added to the surgical robotic system whilst one or more of robot arms 601a-c were operating in the surgical mode 406.

In an example, a new robot arm may be added to a surgical robotic system so as to increase the capabilities of that surgical robotic system. For example, a user of the surgical robotic system (e.g. a surgeon) may desire an additional surgical instrument to aid in a procedure—and so a new robot arm carrying that instrument may be added into the surgical robotic system.

In other examples, a new robot arm may be added to a surgical robotic system in exchange for a robot arm already part of the surgical robotic system. For example, such an exchange may be performed if a robot arm already part of the surgical robotic system develops a fault. That is, a robot arm already part of the surgical robotic system may be removed prior to adding a new robot arm, or a new robot arm may be added to the surgical robotic system prior to removing a robot arm. In order to remove the faulted robot arm from the system, the instrument retract mode described herein may be used. In other words, a robot arm of the surgical robotic system may be replaced by a different robot arm. Examples of faults for which a robot arm may be replaced include mechanical faults (e.g. the failure of a drive cable under mechanical stress), software faults (e.g. the introduction of a software bug), a loss of power from the console, a loss of communication with the console, a loss of communication with the instrument, a casing of the robot arm exceeding predetermined temperature (e.g. caused by a motor of the robot arm overheating), other issues such as a local battery level running low, tracking errors by the motors (e.g. motor slip), problems in the moveable support structure (e.g. a broken wheel, or a brake malfunction) or the case of rail mounted arms, mechanical problems with the rail-arm attachment, or any other fault.

In another example, a robot arm may be removed from the surgical robotic system and then be added back into the same surgical robotic system. In these examples, a robot arm may be removed in order that a maintenance task can be performed (e.g. a fault can be repaired, and/or an alarm can be reset) before that robot arm is added back into the system, or a robot arm may be removed in order that it can be moved into a different location relative to the operating table (e.g. so as to provide better access to a surgical site or avoid collisions with other robot arms) before being added back into the surgical robotic system.

The robot arm described herein could be for purposes other than surgery. For example, the port could be an inspection port in a manufactured article such as a car engine and the robot could control a viewing instrument for viewing inside the engine.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A control system of a surgical robotic system, the surgical robotic system comprising a first robot arm and a second robot arm, each of the first and second robot arms comprising a series of joints by which the configuration of that robot arm can be altered, the series of joints extending from a base at a proximal end of the robot arm to an attachment for a surgical instrument at a distal end of the robot arm, the control system being configured to reconfigure the surgical robotic system by:
controlling the first robot arm to operate in a surgical mode in which a first surgical instrument attached to that first robot arm is inside a patient's body; and
whilst the first robot arm is operating in the surgical mode:
(i) controlling the second robot arm so as to permit a second surgical instrument attached to the second robot arm to be inserted into a port in the patient's body;
(ii) determining a fulcrum about which the second surgical instrument pivots when the configuration of the second robot arm is altered whilst the second surgical instrument is inside the port; and
(iii) controlling the second robot arm to operate in a surgical mode in which the configuration of the second robot arm and second surgical instrument is controlled in response to inputs received at a remote surgeon console whilst maintaining an intersection between the second surgical instrument and the determined fulcrum.

2. The control system as claimed in claim 1, wherein the fulcrum is determined by:
controlling the second robot arm so as to enable its configuration to be altered in response to external forces whilst the second surgical instrument is inside the port; and
determining the fulcrum, the fulcrum being the point about which the surgical instrument of the second robot arm pivots whilst inside the port.

3. The control system as claimed in claim 2, wherein the second robot arm further comprises one or more force sensors configured to sense external forces at one or more joints of the series of joints of the second robot arm, and one or more motors configured to drive one or more joints of the series of joints of the second robot arm, and the control system is further configured to:
control the one or more motors so as to drive one or more joints of the series of joints of the second robot arm in dependence on external forces sensed by the one or more force sensors so as to alter the configuration of the second robot arm.

4. The control system as claimed in claim 1, wherein the second robot arm further comprises one or more position sensors configured to sense the position of one or more joints of the series of joints of the second robot arm and to record the position of one or more joints of the series of joints of the second robot arm at a plurality of instances whilst the configuration of the second robot arm is being altered, and the control system further is configured to:
determine, for each instance, a position of the distal end of the second robot arm in dependence on the respective recorded one or more joint positions;
determine, for each instance, a vector of the second surgical instrument from the determined position of the distal end of the second robot arm in dependence on the respective recorded one or more joint positions; and
determine the point of intersection of the determined vectors of the second surgical instrument so as to determine the fulcrum.

5. The control system as claimed in claim 1, the control system being further configured to:
determine the fulcrum when controlling the second robot arm to operate in a calibration mode; and
control the second robot arm to transition from operating in the calibration mode to operating in the surgical mode.

6. The control system as claimed in claim 5, wherein the second robot arm further comprises a more distal interface and a less distal interface, and the control system is further configured to:
control the second robot arm to transition from operating in the calibration mode to operating in the surgical mode in response to an operator interaction with the more distal interface.

7. The control system as claimed in claim 5, the control system being further configured to:
control the second robot arm so as to permit the second surgical instrument to be inserted into the port by controlling the second robot arm to operate in a compliant mode in which the configuration of the second robot arm can be altered in response to external forces; and
control the second robot arm to transition from operating in the compliant mode to operating in the calibration mode.

8. The control system as claimed in claim 7, the control system being further configured to:
control the second robot arm to transition from operating in the compliant mode to operating in the calibration mode in response to a user interaction with the more distal interface.

9. The control system as claimed in claim 5, the control system being further configured to:
after determining the fulcrum, control the second robot arm to operate in an instrument adjust mode in which the configuration of the second robot arm can be altered in response to external forces but is constrained such that an intersection is maintained between the second surgical instrument and the determined fulcrum.

10. The control system as claimed in claim 9, the control system being further configured to:
control the second robot arm to transition from operating in the calibration mode to operating in the instrument adjust mode;
control the second robot arm to transition from operating in the instrument adjust mode to operating in the surgical mode; and
optionally, control the second robot arm to transition from operating in the surgical mode to operating in the instrument adjust mode.

11. The control system as claimed in claim 1, wherein each of the first robot arm and second robot arm further comprise an orientation interface, and the control system is further configured to:
receive an input identifying a common direction in response to an operator indicating a direction using the orientation interface of the first robot arm and indicating a corresponding direction using the orientation interface of the second robot arm.

12. The control system as claimed in claim 11, wherein, in the surgical mode, the second robot arm is remotely controlled by the control system being configured to:
receive inputs relating to the second robot arm to the remote console;
convert the inputs into control signals for the second robot arm in dependence on the determined fulcrum and the identified common direction; and
control one or more joints of the series of joints of the second robot arm in dependence on the control signals so as to control the configuration of the second robot arm.

13. The control system as claimed in claim 1, wherein the surgical robotic system comprises a third robot arm comprising a series of joints by which the configuration of that robot arm can be altered, the series of joints extending from a base at a proximal end of the robot arm to an attachment for a surgical instrument at a distal end of the robot arm, and the control system is further configured to:
whilst controlling the first robot arm to operate in the surgical mode and prior to permitting the second surgical instrument to be inserted into the port, control the third robot arm so as to permit a third surgical instrument attached to the third robot arm to be retracted from the patient's body.

14. The control system as claimed in claim 13, the control system being further configured to permit the third surgical instrument to be retracted from the patient's body by:
enabling the configuration of the third robot arm to be altered in response to external forces, the freedom of motion of the third robot arm being limited such that the third surgical instrument can only move linearly in directions co-axial with the longitudinal axis of the third surgical instrument and away from the patient's body.

15. The control system as claimed in claim 14, wherein the third robot arm further comprises one or more force sensors configured to sense external forces at one or more joints of the series of joints of the third robot arm, and one or more motors configured to drive one or more joints of the series of joints of the third robot arm, and the control system is further configured to:
resolve external forces sensed by the one or more force sensors so as to determine the components of the forces parallel with the longitudinal axis of the third surgical instrument and away from the patient's body; and
control the one or more motors so as to drive one or more joints of the series of joints of the third robot arm in dependence on the components of the forces parallel with the longitudinal axis of the third surgical instrument so as to alter the configuration of the third robot arm.

16. The control system as claimed in claim 1, wherein the control system is further configured to whilst controlling the first robot arm to operate in the surgical mode and prior to permitting the second surgical instrument to be inserted into the port, control the second robot arm so as to permit the second surgical instrument to be retracted from the patient's body; and control the second robot arm so as to permit the second surgical instrument to be inserted into the patient's body after a maintenance task has been performed on the second robot arm.

17. The control system as claimed in claim 16, the control system being further configured to permit the second surgical instrument to be retracted from the patient's body by:
enabling the configuration of the second robot arm to be altered in response to external forces, the freedom of motion of the second robot arm being limited such that the second surgical instrument can only move linearly in directions parallel with the longitudinal axis of the second surgical instrument.

18. The control system as claimed in claim 17, wherein the second robot arm further comprises one or more force sensors configured to sense external forces at one or more joints of the series of joints of the second robot arm, and one or more motors configured to drive one or more joints of the series of joints of the second robot arm, and the control system is further configured to:
resolve external forces sensed by the one or more force sensors so as to determine the components of the forces parallel with the longitudinal axis of the second surgical instrument; and
control the one or more motors so as to drive one or more joints of the series of joints of the second robot arm in dependence on the components of the forces parallel with the longitudinal axis of the second surgical instrument so as to alter the configuration of the second robot arm.

19. The control system as claimed in claim 1, wherein the surgical mode in which the control system controls the first robot arm to operate is:
an engaged surgical mode in which the configuration of the first robot arm and first surgical instrument is controlled in response to inputs received at the remote surgeon console; or
a disengaged surgical mode in which the configuration of the first robot arm and first surgical instrument is controllable in response to inputs received at the remote surgeon console.

20. A method of reconfiguring a surgical robotic system, the surgical robotic system comprising a first robot arm and a second robot arm, each of the first and second robot arms comprising a series of joints by which the configuration of that robot arm can be altered, the series of joints extending from a base at a proximal end of the robot arm to an attachment for a surgical instrument at a distal end of the robot arm, the method comprising:
operating the first robot arm in a surgical mode in which a first surgical instrument attached to that first robot arm is inside a patient's body; and
whilst operating the first robot arm in the surgical mode:
(i) inserting a second surgical instrument attached to the second robot arm into a port in the patient's body;
(ii) determining a fulcrum about which the second surgical instrument pivots when the configuration of the second robot arm is altered whilst the second surgical instrument is inside the port; and
(iii) operating the second robot arm in a surgical mode in which the configuration of the second robot arm and second surgical instrument is controlled by the remote surgeon console whilst maintaining an intersection between the second surgical instrument and the determined fulcrum.
